(19) United States
(12) Patent Application Publication
(10) Pub. No.: US 2022/0086560 A1
(43) Pub. Date: Mar. 17, 2022

(54) BINAURAL SIGNAL COMPOSING APPARATUS

(71) Applicant: MARGOT WHITE MUSIC LIMITED, London (GB)

(72) Inventor: Margot WEIDEMANN, London (GB)

(73) Assignee: MARGOT WHITE MUSIC LIMITED, London (GB)

(21) Appl. No.: 17/476,118

(22) Filed: Sep. 15, 2021

(30) Foreign Application Priority Data
Sep. 15, 2020 (GB) 2014532.2

(51) Int. Cl. H04R 1/22

(52) U.S. Cl. CPC H04R 1/22

(57) ABSTRACT

A binaural signal composing apparatus is provided to output sound at two discrete frequencies following a single actuation. The apparatus includes an audio output member arranged to output a binaural signal, the binaural signal consisting of a first signal component having a first audio frequency and a second signal component having a second audio frequency. The first audio frequency and the second audio frequency comprise a frequency interval therebetween. The frequency interval being selected from one of: a first frequency interval range; a second frequency interval range; a third frequency interval range; and a fourth frequency interval range. The first, second, third and fourth frequency interval ranges are each selected from between approximately 0 Hz and approximately 25 Hz. The apparatus of the present invention aims to offer a single dedicated apparatus arranged to provide a binaural signal output at frequencies directed at providing improved neural entrainment.

### CROSS REFERENCE TO RELATED APPLICATIONS

This application claims priority benefit from Great Britain Application No. 2014532.2 filed on Sep. 15, 2020, which is incorporated herein by reference in its entirety.

### FIELD OF THE INVENTION

The present invention relates to audible signal composing apparatuses, and particularly to binaural signal composing apparatuses for use in neural entrainment.

### BACKGROUND TO THE INVENTION

Entrainment is acknowledged as a process of synchronising two different systems so that they become harmonious, and is commonly defined by a temporal locking process in which one system's motion or signal frequency entrains the frequency of another system. This process is a universal phenomenon that can be observed in physical (e.g., pendulum clocks) and biological systems (e.g., fire flies). A further loose visualisation of the process involves the concept of sympathetic vibrations. For example, if a musical note is struck in a room in which a guitar is located, the strings of the guitar will vibrate sympathetically to the frequency of the note that was struck.

Entrainment can also be observed between human sensory and motor systems. The function of rhythmic entrainment in therapeutic and rehabilitative training and learning is now well-established, with neural entrainment providing one physiological justification for the placebo effect. Research has shown, for example, that the inherent periodicity of auditory rhythmic patterns may entrain movement patterns in patients with movement disorders.

As an example of therapeutic applications, mathematical models have shown that anticipatory rhythmic templates as critical time constraints can result in the complete specification of the dynamics of a movement over the entire movement cycle, thereby optimizing motor planning and execution. Furthermore, temporal rhythmic entrainment has been successfully extended into applications in cognitive rehabilitation and speech and language rehabilitation, and thus become one of the major neural mechanisms linking music and rhythm to brain rehabilitation. These findings provide a scientific basis for the development of neurologic music therapy.

Earlier forms of neural entrainment have taken place for thousands of years, using forms of meditation accompanying audible stimulation. More recently, neural or brainwave entrainment has involved an assisted form of meditation using sound or light pulses, or both.

There have, until presently, been only limited attempts to explore the utility and feasibility of combining more traditional modes of entrainment with recent advances in technology, and the technical feasibility of such a combination has therefore historically been unclear.

It is therefore desirable to provide a solution which overcomes the drawbacks of current entrainment methods, and particularly which provides a functional combination of traditional entrainment methods with modern understanding.

### SUMMARY OF THE INVENTION

The present application is directed to a signal composing apparatus arranged to output signals, either audibly or by way of an electronic connection to a recording device or memory, comprising two, and only two, discrete frequencies selected from a frequency range, and separated by a predefined frequency interval. Specifically, signal composing apparatuses of the present invention provide an innovative means of investigating and testing the phenomenon of neural entrainment using binaural signals—that is, two auditory signals, each directed to a separate ear of a subject for application to the subject the brain in isolation of one another, the two signals being combined to provide a perceived “binaural beat” occurring at a specific frequency interval between the respective frequencies of the two auditory signals. In most preferable embodiments, the invention permits the user to define a custom array or panel of binaural root frequencies making use of frequency intervals associated with four states of neural entrainment (alpha, beta, delta and theta). The invention is therefore preferably arranged to provide users with a flexible testing platform which may aid in the determination of relationships between the custom root frequency panel and the four established states of neural entrainment. Such a determination may, in some embodiments, be made with the aid of measured outcome variables such as neurological activity. The invention is further intended to provide a flexible, intuitive, compact and efficient mode of defining and establishing a panel of root frequencies suitable for a desired neural entrainment. The present invention may, for example minimise required user input, and make said input, and method of use, easier for an improved overall consumer experience and increased uptake. The invention therefore preferably increases accessibility to equipment suitable for testing and engaging in neural entrainment. In particular embodiments, the invention is directed to a music synthesiser, but other examples of suitable binaural signal composing apparatuses in accordance with the present invention will be envisaged in accordance with the features described herein.

In accordance with particular embodiments of a first aspect of the present invention, a binaural signal composing apparatus is provided, the binaural signal composing apparatus being arranged to output sound at two discrete frequencies following a single actuation, the composing apparatus comprising: an audio output member arranged to output a binaural signal, the binaural signal consisting of: a first signal component having a first audio frequency; and a second signal component having a second audio frequency; wherein the first audio frequency and the second audio frequency comprise a frequency interval therebetween, the frequency interval being selected from one of: a first frequency interval range; a second frequency interval range; a third frequency interval range; and a fourth frequency interval range; and wherein the first, second, third and fourth frequency interval ranges are each selected from between approximately 0 Hz and approximately 25 Hz.

In some preferable embodiments, the first, second, third and fourth frequency ranges may be substantially contiguous, may overlap, or may in some embodiments be non-contiguous and separated.

The frequency interval range of 0 Hz to 25 Hz has been found to be associated with neural entrainment, with sources indicating four entrainment states existing within this range. It has been found that four contiguous frequency interval ranges selected from between 0 Hz to 25 Hz offers an array of desirable neural entrainments eliciting specific responses from subjects preferably defining an objective array of predictable neural outcomes. Most preferable frequency interval ranges may include: an alpha entrainment state between 7 Hz and 13 Hz; a beta entrainment state between 13 Hz and 25 Hz; a delta entrainment state of less than 4 Hz; and a theta entrainment state of between 4 Hz and 7 Hz.

The present invention preferably has the distinct advantage of offering a simple, compact, affordable and efficient solution, providing an apparatus offering a panel of four frequency intervals enabling treatment of a desired audio frequency with the effect of issuing said audio frequency modified for binaural output. The invention offers a single dedicated apparatus arranged to provide a binaural signal output at a frequency directed at providing neural entrainment.

In addition, the present invention may provide: a binaural experience by way of a dual output of the binaural signal to headphones wherein one of the first or second frequency of the binaural signal is issued to an ear of the user, and the other frequency of the binaural signal is issued to another ear of the user; and/or a monaural experience by combining the first and second frequencies of the binaural signal into a single output by way of a loudspeaker, therefore providing a succinct and efficient mode of combining these different outputs. It has been suggested that, while both binaural and monaural terminologies label the same frequency intervallic differences occurring in the binaural signal, different parts of the brain may be affected in each experience of the tones. The present invention, in preferable embodiments, additionally provides an adaptable solution permitting adjustment of a frequency interval, and in addition to this, adjustment of a root frequency to which the frequency interval is applied, the frequency interval and the root frequency having functions which may be effective individually and/or in combination. The present invention also preferably provides an efficient means of combining said frequency interval with the root frequency into an output binaural signal, using a single actuation, thereby providing an efficient mode of providing these frequencies.

The binaural signal composing apparatus is preferably arranged to output: a first binaural signal, the frequency interval of the first binaural signal being selected from the first frequency interval range; a second binaural signal, the frequency interval of the second binaural signal being selected from the second frequency interval range; a third binaural signal, the frequency interval of the third binaural signal being selected from the third frequency interval range; a fourth binaural signal, the frequency interval of the fourth binaural signal being selected from the fourth frequency interval range.

The binaural signal composing apparatus preferably further comprises: an interval selector; and an actuation member; wherein the interval selector is arranged to define a selected frequency interval from one of the first, second, third and fourth frequency interval ranges; and wherein the actuation member is arranged to actuate the audio output member to output the binaural signal according to the selected frequency interval.

Preferably, the first, second, third and fourth frequency interval ranges are substantially discrete relative to one another. Preferably therefore none of the first, second, third and fourth frequency interval ranges substantially overlap with any other of the first, second, third and fourth frequency interval ranges. It will be understood by the skilled addressee that such wording as “substantially discrete” and “substantially overlap” is intended to include any immaterial differences in frequency from those ranges specified. Any immaterial deviation from said ranges is therefore intended to be included. Such immaterial deviation from said ranges may, for example, be around 100 mHz to 500 mHz or less.

In preferable embodiments, the first frequency interval range is greater than approximately 7 Hz and less than approximately 13 Hz; the second frequency interval range is greater than approximately 13 Hz and less than approximately 25 Hz; the third frequency interval range is less than approximately 4 Hz; and the fourth frequency interval range is greater than approximately 4 Hz and less than approximately 7 Hz. Accordingly, the first frequency interval range is therefore, in preferable embodiments, directed to a frequency interval associated with the “alpha” state of neural entrainment; the second frequency interval range is, in preferable embodiments, directed to a frequency interval associated with the “beta” state of neural entrainment; the third frequency interval range is therefore, in preferable embodiments, directed to a frequency interval associated with the “delta” state of neural entrainment; and the fourth frequency interval range is therefore, in preferable embodiments, directed to a frequency interval associated with the “theta” state of neural entrainment.

In most preferable embodiments, the frequency interval is one selected from the group: approximately 4 Hz; approximately 6 Hz; approximately 10 Hz; approximately 17 Hz. It will be understood by the skilled addressee that the term “approximately” used in relation to the prescribed frequency intervals and frequency interval ranges is intended to include immaterial differences from said ranges, such as, for example, any differences of around 100 mHz to 500 mHz or less.

A discussion of the alpha, beta, delta and theta neural entrainment states is provided in the Detailed Description section.

In most preferable embodiments, the first signal frequency and the second signal frequency are selected from one of a plurality of predetermined root frequency ranges. In most preferable embodiments, the predetermined root frequency ranges are selected based on the known optical frequencies of the visible light spectrum, and specifically the known optical frequencies of discrete colours, for example red, orange, yellow, green, blue, indigo and violet. In most preferable embodiments the optical frequencies associated with said colours (in, for example, THz) may undergo a predefined transformation in order to achieve an audio frequency corresponding to said colour, in Hz within an audible range (for example within the human audible range, such as between 20 Hz and 20 kHz). In particular embodiments of the invention, the optical frequencies may undergo a transformation of division by 1 trillion. As an example, the optical frequency associated with the colour red, 405 THz to 480 THz, may undergo a transformation of division by 1 trillion to arrive at an audible root frequency selected from between 405 Hz to 480 Hz. The optical frequencies of any colours, and their corresponding audible root frequencies, will be appreciated, and examples are provided in Table 1 of the Detailed Description section. In accordance with such embodiments of the present invention, the audible root frequency is then subjected to the selected frequency interval by the invention in order to arrive at the first and second frequencies of the first and second components of the binaural signal in accordance with the first aspect. Embodiments will be appreciated wherein any suitable root frequencies may be selected according to a desired research question or outcome. In some embodiments, the first signal frequency and the second signal frequency are freely adjustable by a user, within the selected predetermined root frequency range.

The enhanced customisability of such embodiments of the present invention preferably maximises the utility of the present invention for users intending to practice or investigate the effects of neural entrainment, and specifically to investigate and explore the effects of a combination of modern knowledge of frequency intervals associated with neural entrainment, and any relationship therewith of a custom panel of root frequencies, which may themselves be driven instead by more traditional methods of meditative and neural entrainment teachings. In particular, the present invention preferably allows a user to produce an entirely unique meaning for a frequency produced as a result of the first and second frequencies interacting to provide a binaural beat (for example, by applying a frequency interval associated with an alpha brainwave state to a root frequency associated with the colour orange—commonly associated with the sacral chakra).

The binaural signal composing apparatus preferably comprises a plurality of said actuation members, each said actuation member being mapped to a corresponding root frequency range; and wherein each said actuation member is arranged to actuate the audio output member to output the binaural signal according to the corresponding mapped root frequency range, and the selected frequency interval.

In most preferable embodiments, the plurality of predetermined root frequency ranges may each be one selected from the group: greater than or equal to approximately 430 Hz to 480 Hz; greater than or equal to approximately 480 Hz to 510 Hz; greater than or equal to approximately 510 Hz to 540 Hz; greater than or equal to approximately 540 Hz to 580 Hz; greater than or equal to approximately 540 Hz to 550 Hz; greater than or equal to approximately 550 Hz to 570 Hz; greater than or equal to approximately 570 Hz to 580 Hz; greater than or equal to approximately 580 Hz to 610 Hz; greater than or equal to approximately 610 Hz to 670 Hz; and greater than or equal to approximately 670 Hz to 750 Hz.

Therefore, example embodiments of the present invention may be directed to root frequencies associated with a colour panel, specifically using appropriate transformations of optical frequencies of colours of the panel, to achieve corresponding audio frequencies in the audible range. Such a choice of colours may, for example, be driven by colours associated with a particular emotion or meditative state, such as those associated with traditional yogic or Ayurvedic chakras.

Embodiments may be provided wherein each root frequency range is associated with a respective category of measurable parameters. The root frequencies may therefore be determined according to a desired physical state of a user, and may, for example, be based on historic data attributing said category of measurable parameters to said root frequency range.

In such embodiments, each category of measurable parameters may include one or more selected from the group: heart rate; pulse rate; blood pressure; body temperature; vasodilation; vasoconstriction; blood oxygen concentration; perspiration rate; neural activity; questionnaire results; academic performance; muscle relaxation; measurements of aggressive and/or hostile behaviour; vision perception; athletic performance; attention-deficit hyperactivity disorder; prison reform; epilepsy; lethargy and/or perceived energy levels; otalgia; articular clicking; mandibular deviation. In embodiments employing a questionnaire, the measurable parameter may comprise, for example, emotional state. Embodiments will be appreciated wherein any suitable measurable parameter is used.

Specific outcome measures may be determined in some investigative embodiments of the present invention comprising any suitable means for measuring said parameter, which preferably provides real-time feedback for effective determination of optimal root frequencies for a specific desired outcome. The present invention therefore preferably provides a flexible and reliable determinant of optimal root frequencies for achieving a desired outcome for a particular user.

Each measurable parameter may, in some embodiments, comprise a respective predicted value, each respective predicted value determined according to the associated root frequency range. Embodiments of the present invention may comprise a processor arranged to determine a predicted value of said measurable parameter. In embodiments comprising a suitable means for determining the measurable parameter, a measurement of said parameter during use of the present invention may be compared to the predicted value in order to provide direct feedback to the user.

As such, embodiments of the present invention may provide a simple and compact apparatus for real-time testing and monitoring of effects of the combinations of specific root frequencies with frequency intervals associated with neural entrainment states. Such embodiments may provide a means of identifying optimal root frequencies for a desired outcome, and monitoring such outcomes over time to determine any lasting effects on a user.

In most preferable embodiments, the binaural signal composing apparatus is a music synthesiser. Other embodiments will be appreciated wherein the binaural signal composing apparatus is any composing apparatus suitable for emitting an audio signal for binaural purposes within the scope of the present claims.

Any features described herein as being suitable for incorporation into one or more aspects or embodiments of the present invention, will be understood as being intended to be generalizable across any and all aspects and embodiments of the present disclosure.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 shows a broad schematic view of an example binaural signal composing apparatus, in accordance with an aspect of the present disclosure;

FIG. 2 shows a plan view of an example MIDI controller for use in the binaural signal composing apparatus of FIG. 1, in accordance with an aspect of the present disclosure;

FIG. 3 shows a front view of an example modular rack for use in the binaural signal composing apparatus of FIG. 1, in accordance with an aspect of the present disclosure; and

FIG. 4 shows an oscillogram identifying examples of a first signal component, a second signal component and a combined binaural signal suitable for emission by the present invention, in accordance with an aspect of the present disclosure.

### DETAILED DESCRIPTION FOR CARRYING OUT THE INVENTION

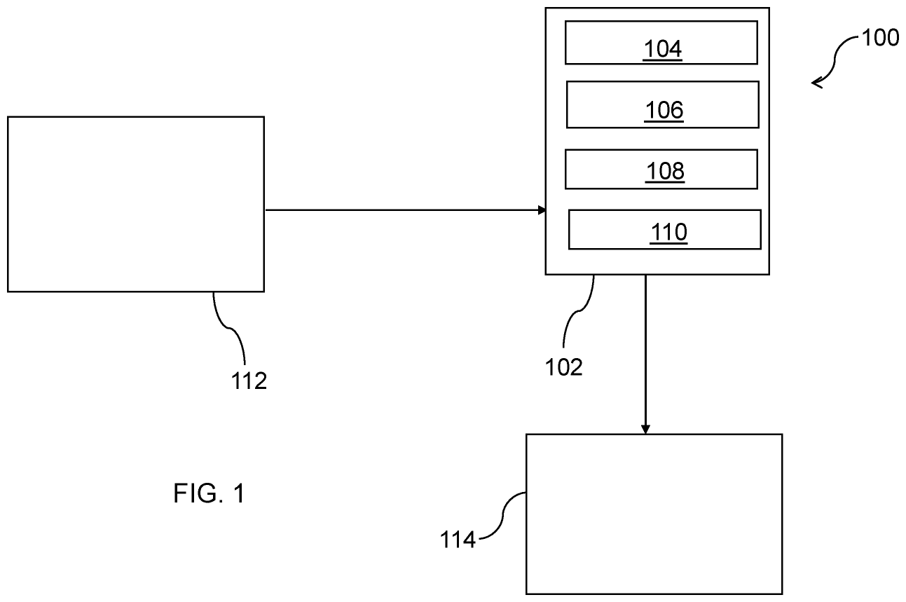

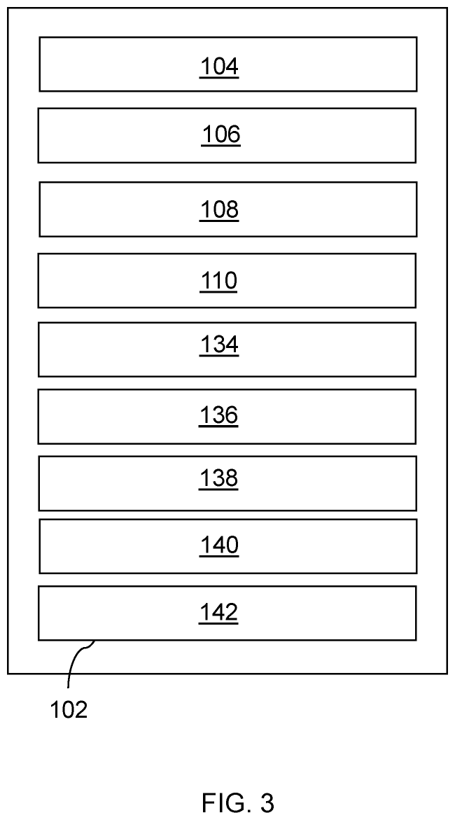

Referring to FIG. 1, a broad schematic view is shown of an example binaural signal composing apparatus 100 in accordance with the first aspect of the present invention. The binaural signal composing apparatus 100 in the example shown takes the form of a music synthesiser 100 and comprises a rack 102 housing a first oscillator 104, a second oscillator 106, a filter 108 and a MIDI interface 110. A more detailed example of a suitable rack 102 layout is described in relation to FIG. 3. The MIDI interface 110 of the modular rack 102 is connected to a MIDI controller 112 (which in the embodiment shown is a MIDI keyboard). The rack 102 comprises an output member arranged to output a stereophonic audio signal to a pair of earphones 114 for binaural purposes. Embodiments will be appreciated wherein the output member is arranged to output the stereophonic audio signal to a recording module or memory. The binaural signal comprises a first channel directed to a first ear of a subject (not shown) and a second, distinct channel directed to the second ear of the subject.

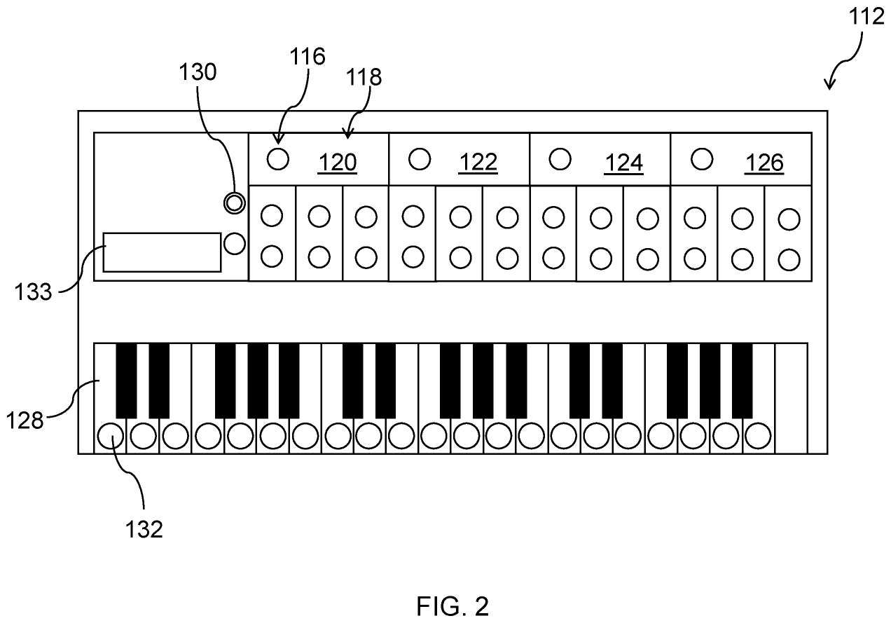

Referring to FIG. 2, a plan view of an example MIDI controller 112 suitable for use in the binaural signal composing apparatus of FIG. 1 is shown. The example MIDI controller 112 is a MIDI keyboard 112 and comprises four primary frequency interval selectors 116, each frequency interval selector 116 associated with a respective frequency interval bank 118.

Of the four frequency interval banks 118 shown, the frequency interval selector of the first bank 120 is arranged to apply a first frequency interval of 10 Hz (or any frequency interval selected between 7 Hz and 13 Hz). The first bank 120 is therefore associated with the “alpha” neural entrainment state. The frequency interval selector of the second bank 122 is arranged to apply a second frequency interval of 17 Hz (or any frequency interval selected between 13 Hz and 25 Hz). The second bank 122 is therefore associated with the “beta” neural entrainment state. The frequency interval selector of the third bank 124 is arranged to apply a third frequency interval of 4 Hz (or any frequency interval selected between 0 Hz and 4 Hz). The third bank 124 is therefore associated with the “delta” neural entrainment state. The frequency interval selector of the fourth bank 126 is arranged to select a fourth frequency interval of 6 Hz (or any frequency interval selected between 4 Hz and 7 Hz). The fourth bank 126 is therefore associated with the “theta” neural entrainment state.

Thereby, the four frequency interval banks 120, 122, 124, 126 each define a single frequency interval within one of the four contiguous but discrete frequency interval ranges associated the four states of neural entrainment existing between 0 Hz and 25 Hz.

Without wishing to be bound by theory, each of the four described neural entrainment states has a unique emotional or cognitive signature. Alpha is related to a softened sense of emotional balance paired with a heightened sense of focus and problem-solving abilities. It is akin to feeling physically relaxed yet extremely mentally alert. This state is used for improving cognitive performance, and is mostly tested on university students, athletes, or anyone wishing to increase their effectiveness in fields of mental agility.

Beta is generally not as desired as the other three states, but it can be helpful in generating growth out of homeostasis of the emotional and energetic system. Additionally, according to a comparative study, beta frequencies used were helpful for improving long term memory and word recall. Beta is related to extreme alertness, and often occurs in moments of extreme stress, as it helps with decision-making. It is best to induce this state with caution and without any prolonged use, as can be the least relaxing of the binaural states.

Theta is an ideal place to start for anyone who is recovering from an accident or trauma such as a car wreck or any physical damage. Additionally it is beneficial for those seeking to heal addictive behaviours. Dr. Gene W. Brockopp notes, “the theta state seems to be the one where behaviour and belief systems change more easily.” Other qualities of the theta state are “integrative experiences leading to feelings of psychological well-being, highly creative and life-altering insights, creating space for ‘super-learning’ in which people are able to learn new languages, accept suggestions for changes in behaviours and attitudes or memorise large amounts of information.” Darren Curtis of Beyond Biofeedback additionally noted there seemed to be a “crossover point at juncture between alpha and theta rhythms accompanied by, ‘the seemingly miraculous resolutions of complex psychological problems.”

Lastly, delta waves seem to be the least studied of all frequencies, and therefore are equally as mysterious as the reported benefits. Running so slowly, between one and four Hz, they are synonymous with a state of deep sleep. This is the state our brain inhabits during the night while the body is restoring its equilibrium and repairing anything calling for attention. A few very interesting points about the delta state can be noted: delta helps us access our subconscious mind, babies spend most of their time in delta, it is the state dominant before death, and delta waves have been link to DHEA production, a hormone that is linked to an increased sense of well-being and cognitive function, although sleep is still the best recourse for DHEA production. The most challenging thing about accessing a primarily delta state during waking hours is alpha and beta waves will be the dominant activity of the brain, and difficult to override. Therefore it may be best to engage with delta frequencies just before sleep or immediately upon waking.

The phenomenon of neural entrainment using the four entrainment states described is generally identified as a Frequency-Following Response, and is not to be confused with a synchronisation of the whole brain. Frequency-Following Response, or FFR, can be monitored through an electroencephalogram (EEG) and is specifically related to “phased sine waves at discernible sound frequencies.” It is primarily effective because it falls in the range of human hearing, is a vibratory frequency that the brain's electrical activity can be sympathetic with. Binaural beats are considered best perceived when the carrier tones are approximately 440 Hz, as anything higher or lower is more difficult to perceive for the human ear. The emotional complexity studies into this field deepens when the parasympathetic nervous system is taken into account. The body naturally chooses equilibrium when it is free of environmental stressors, therefore when these frequencies are able to mimic states of equilibrium in the brain, the body becomes more susceptible to relaxation.

Many studies have shown extremely positive results after prolonged periods during which subjects have been exposed to alpha-, beta-, theta- or delta-enhancing frequencies, and have subsequently achieved success in a variety of areas of focus, such as physical recovery after severe trauma, overcoming lifelong addictions, or cognitive performance enhancement. But there may be an uncontrollable factor that is still to be determined in the field of brainwave or neural entrainment, as the results of the multitude of studies remain generally inconsistent.

This unpredictable variable may be related to some abstract sense of focus on the outcome, or goal, of a prolonged experience with binaural wave entrainment. Another pioneer in the field of binaural brain synchronisation and contemporary of Robert Monroe, Lieutenant F. Holmes At joined Monroe's staff as director of research and contributed further valuable insights. He was one of the first to suggest the use of conscious mental suggestions in correlation with the auditory sensations. He explained, “even though the body is seen as wanting to attain equilibrium, working with levels of autosuggestion and breathing, then close-mapping of the complex brain waves with complex binaural beats, the mind-body connection can come into action.” Other researchers at Hemi-Sync, another branch of binaural research created by Robert Monroe, which offers audio recordings, suggest that “passively listening to Hemi-Sync binaural beats may not automatically engender a focused state of consciousness. The Hemi-Sync process includes a number of components: binaural beats are only one element. We all maintain a psychophysiological momentum, a homeostasis that may resist the influence of the binaural beats. Practices such as humming, toning, breathing exercises, autogenic training, or biofeedback can be used to interrupt the homeostasis of resistant subjects.

Identifying the discrepancies of the results in binaural brainwave entrainment controlled group studies became a crucial point of growth in developing the present invention.

Parallels were drawn between modern schools of schools of thought in the field neural entrainment, and more traditional forms of meditation and focus. Embodiments of the present invention may therefore be directed to an interaction between modern neural entrainment methods operating through the lens of the ancient Sanskrit term, chakra, which translates to “spinning wheel of energy or light” in order to facilitate a strengthened focus when working with binaural entrainment frequencies.

Such an approach could act to complement an apparent need of an additional impetus in the binaural healing process. There are multiple chakras in different disciplines, and the amount of them ranges anywhere from seven to one hundred and fourteen. Multiple cultures reference chakras, but there is still minimal scientific proof of their intricacies.

There is, however, no lack of scientific or academic material documenting electrical impulses governing the body's nervous system, heart and other physiological function.

The present embodiment focuses on nine chakras, labelled here with English and Sanskrit: Root (Muladhara), Sacral (Svadhisthana), Naval/Solar Plexus (Manipura), Lower Heart, Heart (Anahata), Upper Heart, Throat (Vishuddha), Brow/Third-Eye (Ajna), and Crown (Sahasrara).

There are many more that can be found in Indian, Chinese, Tibetan and Japanese teachings, but for the sake of accessibility, the present embodiment attempts to maintain a certain quality of ease for any beginner wishing to investigate or use the present embodiment.

The chosen root frequencies for use in the present embodiment 100 take inspiration from physical understanding of light. In particular, where the optical frequency of the colour red (associated with the Ayurvedic “Root” chakra) is between 405 to 480 THz, the present embodiment makes use of this frequency as a dividend, with the quotient resulting in an audio frequency range of 405 to 480 Hz. This frequency approximates the colour red with the tone ‘A’ for western music assumptions.

In the embodiment shown, the frequency interval selectors 116 take the form of an on/off switch, for example a single pole, single throw switch, arranged to apply the corresponding predetermined or preallocated frequency interval, which is associated with the neural entrainment state of the corresponding bank. Embodiments will be appreciated wherein the frequency interval selectors may be any suitable selector, and may be a continuous selector such as a slider or tuner, or may instead comprise a categorical or discrete bank of one or more frequency interval selector switches each defining a specific frequency interval.

The MIDI controller 112 further comprises a plurality of actuation members 128, the plurality of actuation members 128 in the embodiment 100 shown each taking the form of a keyboard key 128. Each actuation member 128 is arranged to select a respective predefined root frequency to which the selected frequency interval will be applied. Upon pressing of a key 128 by a user, each said key/actuation member 128 is arranged to cause the output of a corresponding signal by an output member 130 of the MIDI controller 112 to the MIDI interface 110 of the rack 102.

The MIDI keyboard 112, in the embodiment shown is a three-octave keyboard controller. The chosen frequency interval bank determines an associated collection of frequency intervals as pitches to be applied to a root frequency selected according to a pressed key 128, the resulting frequencies being output by the MIDI keyboard 112 when the keys 128 of the keyboard 112 are pressed. Once a selection of a frequency interval bank 118 is made by way of the corresponding frequency interval selector 116, the MIDI keyboard 112 offers the user a choice of root frequencies in three octaves, to which the selected frequency interval will be applied.

In addition, the embodiment shown comprises a “traditional synth” mode selector (not shown) where upon selection, the keyboard 112 performs the functions of a traditional pitch generator, thus offering singular frequencies attributed to each key 128 on the keyboard 112. Such a feature provides flexibility to the user in the functions performs by the embodiment.

In the embodiment shown, the frequency interval selectors 116 take the form of a toggle switch, but embodiments will be appreciated wherein the frequency interval selectors will be selected via an LCD or OLED screen, or any other suitable selection mechanism.

Each key 128 of the MIDI keyboard 112 pressed by the user is arranged to trigger two frequencies simultaneously (to be generated by the oscillators 104, 106 of the rack 102), with the interval between the two frequencies being dictated by the frequency interval bank 118 chosen by the user. In the embodiment shown, the keys 128 each comprise a symbol 132 representing a corresponding root frequency embossed thereon, allowing the user to locate a desired root frequency. The keys 128 will always trigger an output associated with the same root frequency, irrespective of the frequency interval bank 118 selected, thereby allowing the user to explore the effects of the root frequency when different frequency interval banks 118 are applied thereto. In the embodiment shown, the root frequencies are associated with yogic, Vedic or Ayurvedic chakras, each key having an associated chakra symbol embossed thereon.

Whenever a key 128 of the MIDI keyboard 112 is pressed, an electrical tuning signal corresponding to the root frequency of the key 112, and the selected frequency interval, is produced and output by the MIDI keyboard 112. The electric tuning signal comprises a first tuning signal associated with the first frequency output by way of a first output 130 of the MIDI keyboard 112 to the first voltage controlled oscillator 104, and a second tuning signal associated with the second frequency by way of a second output 131 of the MIDI keyboard 112 to the second voltage controlled oscillator 106.

Referring to FIG. 3, an exemplary version of a suitable rack 102 is shown, the MIDI keyboard 112 (of FIG. 1 and FIG. 2) being connected to the rack 102 through a MIDI interface 110 (Yarns MIDI Interface by Mutable Instruments), two voltage controlled oscillators 104, 106 (Instruo Tona Oscillators), a voltage controlled amplifier 134 (Deeper A130-2 Voltage Control Amplifier), a voltage controlled filter 108 (Ripples filter by Mutable Instruments which also delivers a clean sine tone through the LP4 output), a low-frequency oscillator 136 (Maths Analog LFO by MakeNoise), a master clock with multimode divider 138 (Horologic Solum Master Clock & Multi Mode Divider), a mixer module 140 (Doepfer A-138n Mixer Module) and a sequencer 142 (Intellijel Scales Sequencer). Combined, these units form the music synthesizer 100 of the example embodiment. Following a single actuation of an actuation member 116, a signal is provided to the MIDI interface 110 indicating two corresponding pitches (for example as set out in Table 1). The two pitches, having a defined frequency according to the selected frequency interval, are then processed through the MIDI interface 110 to engage with the rest of the modules of the rack 102, producing a binaural signal.

Each of the first and second voltage controlled oscillators 104, 106 are “triggered” by the respective tuning signals received from the MIDI keyboard 112 using a traditional one volt per octave system. Each tuning signal is received by the corresponding voltage controlled oscillator 104, 106 and used by the voltage controlled oscillator to tune the voltage controlled oscillator to the desired frequency. Each of the voltage controller oscillators 104, 106 provide a corresponding transformation to the tuning signal, to provide an output at a specified waveshape (sine, triangle, or saw; and noise, or pulse).

The output signals from the respective voltage controlled oscillators 104, 106 are received by voltage controlled filter 108 of the rack 102 in the embodiment shown, which contours the frequency spectrum of the waves received from each voltage controlled oscillator 104, 106. The voltage controlled filter 108 often affects more the aesthetic/tone quality of the wave. This quality varies according to the voltage control filter module chosen, and suitable versions will be appreciated. The voltage controlled filter is one module of the rack 102 which affects the final binaural signal most drastically.

The signal then passes to a voltage controlled amplifier 134 of the rack 102, which controls a volume of the generated and filtered waveform over time. An “envelope” is applied to the voltage controlled amplifier 134, and is a term used to describe the “shape” of the volume of a sound over time, allowing a user to set the start and end points of an output audio signal. Commonly the envelope contains four parameters: attack; sustain; decay; and release (commonly referred to using the initialism ADSR). “Attack” describes a time between silence and the initial loudest point, while “decay” describes a time for the envelope to decrease after the initial loudest point, to a steady value. The sound then continues at a level known as the “sustain”, and remains at this level for as long as the key 112 is held down. Once the key 112 is released, the sound resumes its decay, this time at a rate determined by the “release” setting.

The embodiment shown also comprises additional features which may be employed by a user according to a desired effect achieved on the binaural signal, such as modules by specific manufacturers enabling unique proprietary effects. Suitable modules will be appreciated by the skilled addressee. In any embodiments, the frequencies may, at some point in any suitable embodiment, be able to be manipulated at the stage of the voltage controlled filter, for example in order to produce different timbres of the same frequency.

One module of the presently-described embodiment is shown for such illustration purposes only, which is a low frequency oscillator 136 which can be used to manipulate tones in an extraneous way in accordance with the user's wishes, such as for modulating tones backwards for example. This and other modules may additionally be used for modulating the frequencies to produce ‘wobbly’ audio effects such as tremolo.

The rack 102 is arranged to output the binaural signal comprising the first frequency and the second frequency by way of an output member on the mixer.

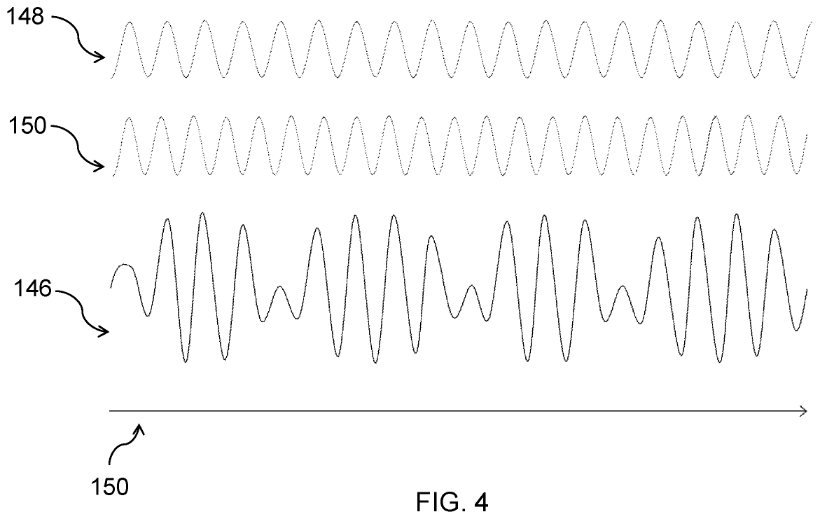

The binaural signal is described in more detail in relation to FIG. 4 and comprises two signal components emitted separately by the output member. The output member in the embodiment shown comprises a 3.5 mm audio jack output arranged to transmit the binaural signal in a stereoscopic manner to earphones 114, each frequency component of the binaural signal being transmitted on a separate channel of said earphones 114, each to be detected by a separate ear of the user.

Each actuation member 128 of the MIDI controller 112 in the embodiment shown comprises a label 132 indicating the respective root frequency of said actuation member 128, to which the selected frequency interval is to be applied to provide a corresponding binaural signal.

The embodiment shown represents an example binaural composing apparatus 100 in accordance with the present invention, which provides a user with a simple and compact tool to investigate the effects of combining specific root frequencies with neural entrainment state-associated frequency intervals for neural entrainment purposes. In particular, the example shown makes use of a panel of root frequencies associated with traditional yogic, Vedic or Ayurvedic meditation.

The nine chakras commonly studied in yogic and Ayurvedic traditions correlate to colours, and so the present embodiment uses these accepted colours to build the tonality of the root frequencies. Therefore, as outlined in Table 1 below, the plurality of root frequencies, each allocated to a corresponding actuation member 128, are determined according to an optical frequency value of colours commonly associated with respective chakras, each transformed to an audio frequency value through a division by 1 trillion.

TABLE 1
binaural signal components of the present embodiment 100 of FIG. 1 to
FIG. 4 according to root frequency and selected frequency interval

| | Alpha | Beta | Theta | Delta |
|---|---|---|---|---|
| | 7 Hz to | 13 Hz to | 4 Hz to | less than |
| | 13 Hz | 25 Hz | 7 Hz | 7 Hz |
| Frequency Interval | 10 Hz | 17 Hz | 6 Hz | 4 Hz |
| Root chakra (red) | 460 Hz and | 461 Hz and | 444 Hz and | 444 Hz and |
| 430 Hz to 480 Hz | 450 Hz | 444 Hz | 438 Hz | 440 Hz |
| Sacral chakra (orange) | 500 Hz and | 507 Hz and | 498 Hz and | 490 Hz and |
| 480 Hz to 510 Hz | 490 Hz | 490 Hz | 492 Hz | 486 Hz |
| Solar Plexus chakra (yellow) | 530 Hz and | 529 Hz and | 536 Hz and | 530 Hz and |
| 510 Hz to 540 Hz | 520 Hz | 512 Hz | 530 Hz | 526 Hz |
| Lower Heart chakra (green) | 550 Hz and | 559 Hz and | 558 Hz and | 548 Hz and |
| 540 Hz to 550 Hz | 540 Hz | 542 Hz | 552 Hz | 544 Hz |
| Heart chakra (green) | 565 Hz and | 569 Hz and | 566 Hz and | 562 Hz and |
| 550 Hz to 570 Hz | 555 Hz | 552 Hz | 560 Hz | 558 Hz |
| Upper Heart chakra (green) | 580 Hz and | 589 Hz and | 576 Hz and | 578 Hz and |
| 570 Hz to 580 Hz | 570 Hz | 572 Hz | 570 Hz | 574 Hz |
| Throat chakra (blue) | 600 Hz and | 599 Hz and | 606 Hz and | 602 Hz and |
| 580 Hz to 610 Hz | 590 Hz | 582 Hz | 600 Hz | 598 Hz |
| Third Eye chakra (indigo) | 650 Hz and | 646 Hz and | 668 Hz and | 666 Hz and |
| 610 Hz to 670 Hz | 640 Hz | 629 Hz | 662 Hz | 662 Hz |
| Crown chakra (violet) | 710 Hz and | 689 Hz and | 715 Hz and | 696 Hz and |
| 670 Hz to 750 Hz | 700 Hz | 672 Hz | 709 Hz | 692 Hz |

The MIDI keyboard 112 further comprises a display screen 133 positioned to signal, for example, an actuation event to a user.

Referring to FIG. 4, an oscillogram trace 144 is provided outlining an example binaural signal 146 comprising a first signal component 148 operating at a first frequency and a second signal component 150 operating at a second frequency, the first frequency being separated from the second frequency by one of the four selectable frequency intervals according to a desired neural entrainment state. The first frequency and the second frequency are determined based on a root frequency selected according to a desired meditative focus, which in the specific example provided, comprises a series of yogic, Vedic or Ayurvedic meditation states or “chakras”. Examples of suitable first and second signal components 148, 150 are outlined in Table 1.

It will be appreciated that the above described embodiments are given by way of example only and that various modifications may be made to the described embodiments without departing from the scope of the invention as defined in the appended claims.

As may be recognized by those of ordinary skill in the art based on the teachings herein, numerous changes and modifications may be made to the above-described and other embodiments of the present disclosure without departing from the scope of the disclosure. The components of the invention as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. Accordingly, this detailed description of the currently-preferred embodiment is to be taken in an illustrative, as opposed to limiting of the disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations

1. A binaural signal composing apparatus arranged to output sound at two discrete frequencies following a single actuation, the composing apparatus comprising:
an audio output member arranged to output a binaural signal, the binaural signal consisting of:
a first signal component having a first audio frequency; and
a second signal component having a second audio frequency;
wherein the first audio frequency and the second audio frequency comprise a frequency interval therebetween, the frequency interval being selected from at least one of:
a first frequency interval range;
a second frequency interval range;
a third frequency interval range; and
a fourth frequency interval range; and
wherein the first, second, third and fourth frequency interval ranges are each selected from between approximately 0 Hz and approximately 25 Hz.

2. The binaural signal composing apparatus of claim 1, wherein the binaural signal composing apparatus is arranged to output:
a first binaural signal, the frequency interval of the first binaural signal being selected from the first frequency interval range;
a second binaural signal, the frequency interval of the second binaural signal being selected from the second frequency interval range;
a third binaural signal, the frequency interval of the third binaural signal being selected from the third frequency interval range; and/or
a fourth binaural signal, the frequency interval of the fourth binaural signal being selected from the fourth frequency interval range.

3. The binaural signal composing apparatus of claim 1, wherein the binaural signal composing apparatus further comprises:
an interval selector; and
an actuation member;
wherein the interval selector is arranged to define a selected frequency interval from one of the first, second, third and fourth frequency interval ranges; and
wherein the actuation member is arranged to actuate the audio output member to output the binaural signal according to the selected frequency interval.

4. The binaural signal composing apparatus of claim 3, wherein the first signal frequency and the second signal frequency are selected from one of a plurality of predetermined root frequency ranges.

5. The binaural signal composing apparatus of claim 4, wherein the first signal frequency and the second signal frequency are freely adjustable by a user, within the selected predetermined root frequency range.

6. The binaural signal composing apparatus of claim 4, wherein the binaural signal composing apparatus comprises:
a plurality of said actuation members, each said actuation member being mapped to a corresponding root frequency range; and
wherein each said actuation member is arranged to actuate the audio output member to output the binaural signal according to the corresponding mapped root frequency range, and the selected frequency interval.

7. The binaural signal composing apparatus of claim 4, wherein the plurality of predetermined root frequency ranges is each selected from at least one of the group comprising:
greater than or equal to approximately 430 Hz to 480 Hz;
greater than or equal to approximately 480 Hz to 510 Hz;
greater than or equal to approximately 510 Hz to 540 Hz;
greater than or equal to approximately 540 Hz to 580 Hz;
greater than or equal to approximately 540 Hz to 550 Hz;
greater than or equal to approximately 550 Hz to 570 Hz;
greater than or equal to approximately 570 Hz to 580 Hz;
greater than or equal to approximately 580 Hz to 610 Hz;
greater than or equal to approximately 610 Hz to 670 Hz; and
greater than or equal to approximately 670 Hz to 750 Hz.

8. The binaural signal composing apparatus of claim 4, wherein each root frequency range associated with a category of measurable parameters.

9. The binaural signal composing apparatus of claim 8, wherein each category of measurable parameters includes one or more selected from the group consisting of: heat rate; pulse rate; blood pressure; body temperature; vasodilation; vasoconstriction; blood oxygen concentration; perspiration rate; neural activity.

10. The binaural signal composing apparatus of claim 8, wherein each measurable parameter comprises a respective predicted value, each respective predicted value determined according to the associated root frequency range.

11. The binaural signal composing apparatus of claim 10, wherein the first, second, third and fourth frequency interval ranges are substantially discrete relative to one another.

12. The binaural signal composing apparatus of claim 1, wherein:
the first frequency interval range is greater than approximately 7 Hz and less than approximately 13 Hz;
the second frequency interval range is greater than approximately 13 Hz and less than approximately 25 Hz;
the third frequency interval range is less than approximately 4 Hz; and
the fourth frequency interval range is greater than approximately 4 Hz and less than approximately 7 Hz.

13. The binaural signal composing apparatus of claim 12, wherein the frequency interval is one selected from the group consisting of: approximately 4 Hz; approximately 6 Hz; approximately 10 Hz; approximately 17 Hz.

14. The binaural signal composing apparatus of claim 1, wherein the binaural signal composing apparatus is a music synthesiser.
